SEQUENCE LISTING (1 sequence)

SEQ ID NO: 1 - DNA, 2869 bp, Mus Musculus
gccgcgaccagccaggccggccagtcgggctcagcccggagacagtcgagacccctgactgcagcaggatggctcagtggaaccagctgcagcagctggacacacgctacctgaagcagctgcaccagctgtacagcgacacgttccccatggagctgcggcagttcctggcaccttggattgagagtcaagactgggcatatgcagccagcaaagagtcacatgccacgttggtgtttcataatctcttgggtgaaattgaccagcaatatagccgattcctgcaagagtccaatgtcctctatcagcacaaccttcgaagaatcaagcagtttctgcagagcaggtatcttgagaagccaatggaaattgcccggatcgtggcccgatgcctgtgggaagagtctcgcctcctccagacggcagccacggcagcccagcaagggggccaggccaaccacccaacagccgccgtagtgacagagaagcagcagatgttggagcagcatcttcaggatgtccggaagcgagtgcaggatctagaacagaaaatgaaggtggtggagaacctccaggacgactttgatttcaactacaaaaccctcaagagccaaggagacatgcaggatctgaatggaaacaaccagtctgtgaccagacagaagatgcagcagctggaacagatgctcacagccctggaccagatgcggagaagcattgtgagtgagctggcggggctcttgtcagcaatggagtacgtgcagaagacactgactgatgaagagctggctgactggaagaggcggcagcagatcgcgtgcatcggaggccctcccaacatctgcctggaccgtctggaaaactggataacttcattagcagaatctcaacttcagacccgccaacaaattaagaaactggaggagctgcagcagaaagtgtcctacaagggcgaccctatcgtgcagcaccggcccatgctggaggagaggatcgtggagctgttcagaaacttaatgaagagtgccttcgtggtggagcggcagccctgcatgcccatgcacccggaccggcccttagtcatcaagactggtgtccagtttaccacgaaagtcaggttgctggtcaaatttcctgagttgaattatcagcttaaaattaaagtgtgcattgataaagactctggggatgttgctgccctcagagggtctcggaaatttaacattctgggcacgaacacaaaagtgatgaacatggaggagtctaacaacggcagcctgtctgcagagttcaagcacctgacccttagggagcagagatgtgggaatggaggccgtgccaattgtgatgcctccttgatcgtgactgaggagctgcacctgatcaccttcgagactgaggtgtaccaccaaggcctcaagattgacctagagacccactccttgccagttgtggtgatctccaacatctgtcagatgccaaatgcttgggcatcaatcctgtggtataacatgctgaccaataaccccaagaacgtgaacttcttcactaagccgccaattggaacctgggaccaagtggccgaggtgctcagctggcagttctcgtccaccaccaagcgagggctgagcatcgagcagctgacaacgctggctgagaagctcctagggcctggtgtgaactactcagggtgtcagatcacatgggctaaattctgcaaagaaaacatggctggcaagggcttctccttctgggtctggctagacaatatcatcgaccttgtgaaaaagtatatcttggccctttggaatgaagggtacatcatgggtttcatcagcaaggagcgggagcgggccatcctaagcacaaagcccccgggcaccttcctactgcgcttcagcgagagcagcaaagaaggaggggtcactttcacttgggtggaaaaggacatcagtggcaagacccagatccagtctgtagagccatacaccaagcagcagctgaacaacatgtcatttgctgaaatcatcatgggctataagatcatggatgcgaccaacatcctggtgtctccacttgtctacctctaccccgacattcccaaggaggaggcatttggaaagtactgtaggcccgagagccaggagcaccccgaagccgacccaggtagtgctgccccgtacctgaagaccaagttcatctgtgtgacaccaacgacctgcagcaataccattgacctgccgatgtccccccgcactttagattcattgatgcagtttggaaataacggtgaaggtgctgagccctcagcaggagggcagtttgagtcgctcacgtttgacatggatctgacctcggagtgtgctacctcccccatgtgaggagctgaaaccagaagctgcagagacgtgacttgagacacctgccccgtgctccacccctaagcagccgaaccccatatcgtctgaaactcctaactttgtggttccagatttttttttttaatttcctacttctgctatctttgggcaatctgggcactttttaaaagagagaaatgagtgagtgtgggtgataaactgttatgtaaagaggagagacctctgagtctggggatggggctgagagcagaagggaggcaaaggggaacacctcctgtcctgcccgcctgccctcctttttcagcagctcgggggttggttgttagacaagtgcctcctggtgcccatggctacctgttgccccactctgtgagctgataccccattctgggaactcctggctctgcactttcaaccttgctaatatccacatagaagctaggactaagcccaggaggttcctctttaaattaaaaaaaaaaaaaaaa

(19) United States
(12) Patent Application Publication
(10) Pub. No.: US 2007/0213288 A1
(43) Pub. Date: Sep. 13, 2007

(54) Adenoviral Vector Capable of Infecting Tumor Cells and Eliminating the Function of STAT3

(75) Inventors: Eric B. Haura, Tampa, FL (US); Richard Jove, Tampa, FL (US); Tammy Bowman, Tampa, FL (US); Lanxi Song, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/565,287

(22) Filed: Nov. 30, 2006

Related U.S. Application Data
(62) Division of application No. 10/894,200, filed on Jul. 19, 2004.
(60) Provisional application No. 60/481,105, filed on Jul. 17, 2003.

(51) Int. Cl. A61K 31/7052; C07H 21/04

(52) U.S. Cl. 514/44; 536/23.5

(57) ABSTRACT

An adenoviral vector which expresses a dominant negative form of Stat3 called Stat3-EVA for the treatment of non-small cell lung carcinoma. This construct has two mutations in the DNA binding site of Stat3 which prevents binding to DNA but has no effect on tyrosine phosphorylation or dimerization.

### CROSS REFERENCE TO RELATED APPLICATIONS

This application is a Divisional application of co-pending U.S. patent application Ser. No. 10/894,200, filed Jul. 19, 2004; said application claims priority to U.S. Provisional Patent Application No. 60/481,105, entitled, “Ad-Stat3EVA”, filed Jul. 17, 2003.

### BACKGROUND OF THE INVENTION

Carcinoma of the lung continues to be the largest killer of Americans due to cancer—lung cancer kills more Americans each year than all deaths due to breast, colon, and prostate cancer combined. A large body of work has implicated overexpression of receptor tyrosine kinases, such as the epidermal growth factor receptor, as well as non-receptor tyrosine kinases, such as Src, in the formation of human lung cancers. The activation of receptor tyrosine kinases , non-receptor kinases like Src, and cytokine receptors leads to activation of the STATpathway. STATs are latent cytoplasmic transcription factors which form dimers when phosphorylated and translocate to the nucleus to regulate expression of genes important in cell growth and survival.

While no direct studies into the role of STATs in human lung cancer have been undertaken, a large body of evidence points to their potential importance. Tyrosine kinase growth factor receptors are over-expressed in a large number of human lung cancers, with non-small cell lung cancers demonstrating overexpression of EGF-R and c-Met, while some small cell lung cancers demonstrate c-Kit overexpression. Src, a major upstream regulator of STAT activity, has also been suggested to be activated in human lung cancers. One study has suggested a role for STAT3 activation in a lung cancer cell line transformed by HER2/c-erbB2. IL-6, a known upstream regulator of STAT signal transduction acting through the gpl3O receptor, has been found to be elevated in nearly 40% of lung cancer patients.

Overexpression of receptor tyrosine kinases including the epidermal growth factor receptor (EGF-R) as well as non-receptor tyrosine kinases, such as Src, have been implicated in the formation of human lung cancers. In addition, cytokines like interleukin-6 (IL-6) have been demonstrated to modulate lung cancer cell growth and elevated levels of IL-6 have been shown to be an adverse prognostic factor for patients with lung cancer. Despite a large body of evidence pointing to their potential importance, few direct studies into the role of Signal Transducers and Activators of Transcription (STAT) pathways in human lung cancer have been undertaken.

### SUMMARY OF INVENTION

One embodiment of the present invention includes a method of treating cancer in a patient comprising the steps of providing an adenoviral vector which expresses a dominant-negative of Stat3 and transfecting a target cell with the adenovirus vector. In this embodiment the cancer is non-small-cell lung cancer and the target cell is a cancer cell. The adenoviral vector comprises at least one mutation in the DNA-binding-site of SEQ ID NO 1. The at least one mutation in the DNA-binding-site of SEQ ID NO 1 further comprises the mutation of amino acids 343 and 435 from glutamic acid to alanine. Additionally, in this embodiment, another mutation in the DNA-binding-site of SEQ ID NO 1 includes the mutation of amino acids 461 through 463 from valine acid to alanine. Although this mutation can be achieved by any method known in the art, this embodiment includes the use of Polymerase-Chain-Reaction mutagenesis.

In another embodiment of the present invention, an adenoviral vector is provided which which expresses a dominant negative form of stat3, comprising at least one mutation in the DNA-binding-site of SEQ ID NO 1 and a shuttle vector. The adenoviral vector comprises the mutation of amino acids 434 and 435 from glutamic acid to alanine. Additionally, in this embodiment, another mutation in the DNA-binding-site of SEQ ID NO 1 includes the mutation of amino acids 461 through 463 from valine acid to alanine. Although this mutation can be achieved by any method known in the art, this embodiment includes the use of Polymerase-Chain-Reaction mutagenesis. Finally, the shuttle vector comprises an AdTrack-CMV Plasmid.

### BRIEF DESCRIPTION OF THE DRAWINGS

For a fuller understanding of the invention, reference should be made to the following detailed description, taken in connection with the accompanying drawings, in which:

FIG. 1 shows A549 cells which were infected with either Ad-GFP or Ad-GFP-Stat3-EVA as described and harvested 24 hours later and nuclear extracts were prepared for EMSA. Control cells were mock-infected and received no adenovirus. Numbers after virus infection refer to multiplicity of infection (MOI).

FIG. 2 shows photomicrographs of A549 cells 96 hours after infection with Ad-GFP or Ad-GFP-Stat3-EVA.

FIG. 3 shows A549 cells which were collected 24 hours after infection and prepared for EMSA analysis for Stat3 in addition to total protein for PARP analysis.

FIG. 4 is a diagrammatic representation of the inventive method wherein amino acids 434 and 435 are mutated from glutamic acid to alanine and amino acids 461 through 463 are mutated from valine to alanine via PCR mutagenesis.

FIG. 5 is a diagrammatic representation of the inclusion of the Stat3-EVA fragment with the shuttle vector.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

Here, the inventors demonstrate that multiple non-small cell lung cancer cell lines demonstrate constitutive Stat3 DNA-binding activity. Stat3 DNA-binding activity is specifically upregulated by the addition of epidermal growth factor (EGF), IL-6, and hepatocyte-derived growth factor (HGF). Furthermore, the stimulation of Stat3 DNA-binding activity by EGF requires the activity of EGF-R tyrosine kinase as well as Src-kinase, while the upregulation of Stat3 activity by IL-6 or HGF requires only Src-kinase activity. Treatment of A549 lung cancer cells with PD 180970 or SU6656, both pharmacological inhibitors of Src-kinase, resulted in reduced Src and Stat3 activity, cell cycle arrest in G2, and reduced viability of cells accompanied by induction of apoptosis. Treatment of Stat3 positive A549 and H358 cells with antisense Stat3 oligonucleotides results in complete loss of Stat3 DNA-binding activity and apoptosis, while Stat3 positive H 1299 cells remained healthy. Finally, an adenoviral vector expressing a dominant negative Stat3 isoform results in loss of Stat3 DNA binding activity, apoptosis, and reduced cellular viability. These results demonstrate a role of Stat3 in transducing survival signals downstream of tyrosine kinases such as Src, EGF-R and c-Met, as well as cytokines such as IL-6, in human non-small cell lung cancers.

Adenoviral Delivery of Dominant Negative Stat3 Induces Apoptosis in A549 Cells

The inventive adenoviral vector expresses a dominant negative form of Stat3 (SEQ ID NO. 1) called Stat3-EVA. This construct has two mutations in the DNA binding site of Stat3 which prevents binding to DNA but has no effect on tyrosine phosphorylation or dimerization. A549 cells were grown and infected with two multiplicities of infection (MOI) and controls consisted of cells infected with adenovirus expressing green fluorescent protein (GFP). Twenty-four hours after infection the cells were harvested and nuclear extracts prepared for Electrophoretic Mobility Shift Assay (EMS). FIG. 1 demonstrates loss of Stat3 DNA binding when cells were infected with 25 or 50 MOI of Ad Stat3-EVA but no inhibition of DNA binding was seen with cells infected with Ad-GFP at equivalent viral doses. Infection of cells with Ad-Stat3-EVA also resulted in a dose-dependent reduction in Stat3 DNA binding activity (data not shown). Cells were infected at MOI of 5, 10, 25, and 50 and EMSA was performed after 36 hours of infection. Infection with a MOI of 5 resulted in nearly 90% reduction in Stat3 activity while higher doses were associated with near complete loss of Stat3 activity assayed by EMSA.

### EXAMPLE 1

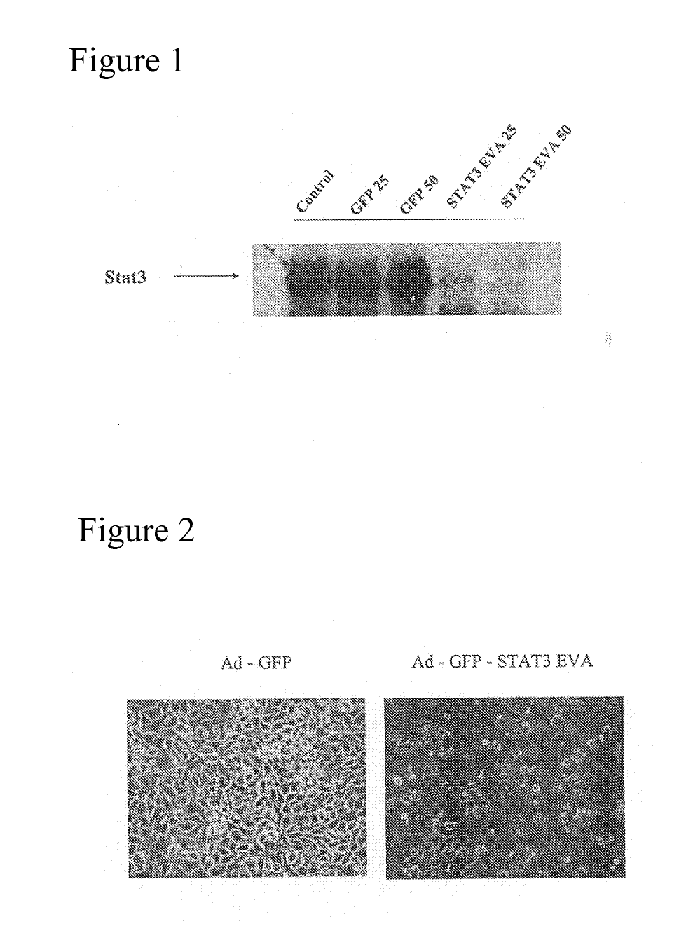

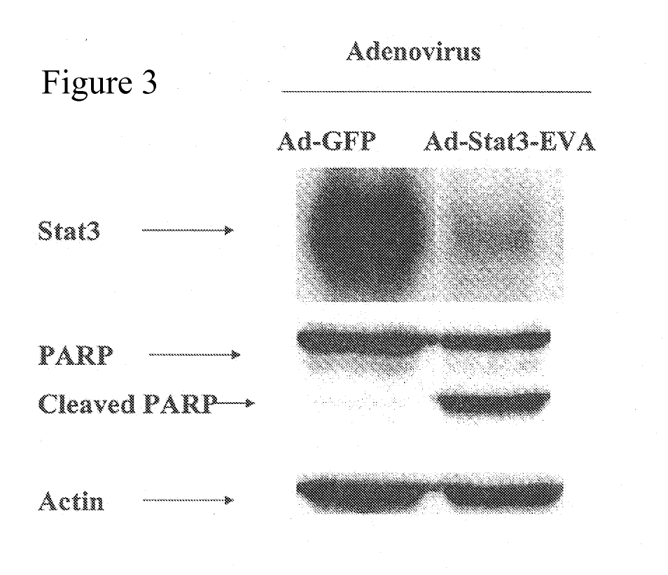

A549 cells were treated with Ad-Stat3-EVA or Ad-GFP at a MOI of 10 and observed the cells in culture. After approximately 48 hours of infection, cells infected with Ad-Stat3-EVA became rounded, refractile, and began to float and at 96 hours after infection widespread cell death was apparent in the cells infected with Ad-Stat3-EVA but minimal effects were seen with cells infected with Ad-GFP FIG. 2. Note however that one cannot assay for apoptosis using apo-BrdU incorporation since the FITC-labeled antibody and GFP expressed by the adenoviral vector overlap in fluorescence. However, cells infected with Ad-Stat3-EVA demonstrated PARP cleavage indicative of apoptosis, while cells infected with Ad-GFP did not demonstrate PARP cleavage FIG. 3. PARP, as used herein refers to a 116 kDa nuclear protein which is strongly activated by DNA strand breaks. PARP plays a role in DNA repair as well as in other cellular processes, including DNA replication, cell proliferation and differentiation. During apoptosis, ICE family members, such as caspase 3 and 7, cleave PARP to yield an 85 kDa and a 25 kDa fragment. PARP cleavage is considered to be one of the classical characteristics of apoptosis.

Finally, cells infected with Ad-Stat3-EVA demonstrated reduced viability assayed by trypan blue staining while cells infected with Ad-GFP had no significant affect on cellular viability (FIG. 3). Therefore, the by using a dominant negative Stat3 adenovirus, it has been shown that loss of Stat3 function in human alveolar epithelial (A549) lung carcinoma cells results in cell death through apoptosis.

Adenoviral Vectors

Therefore, provided is an adenoviral vector which express a dominant negative form of Stat3 termed Stat3-EVA. This mutant form of Stat3 fails to bind DNA and therefore acts as a dominant negative form of Stat3.

### EXAMPLE 2

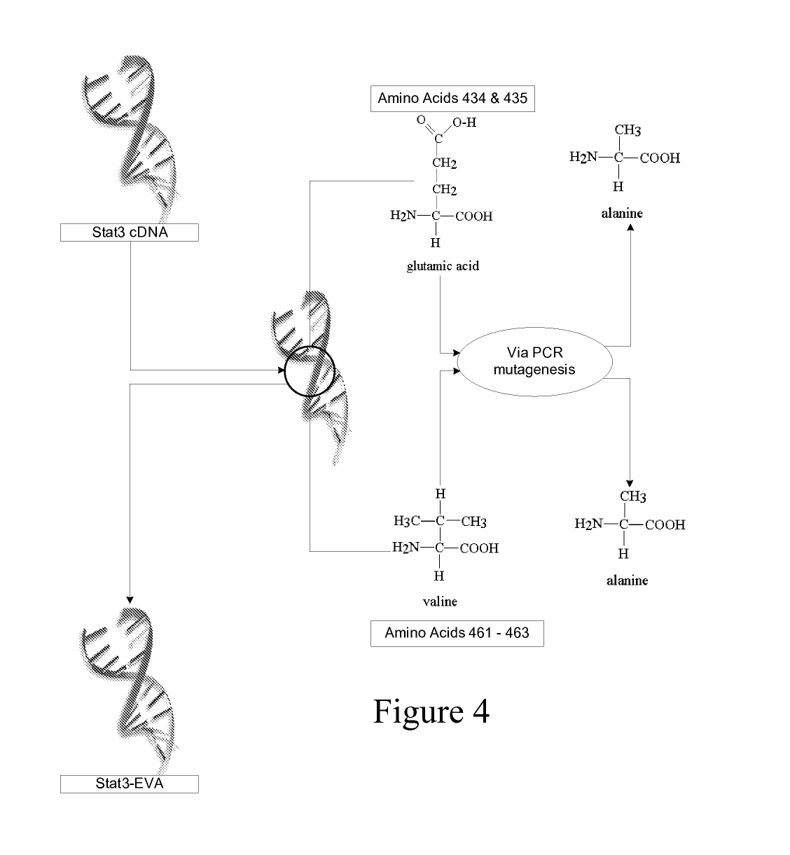

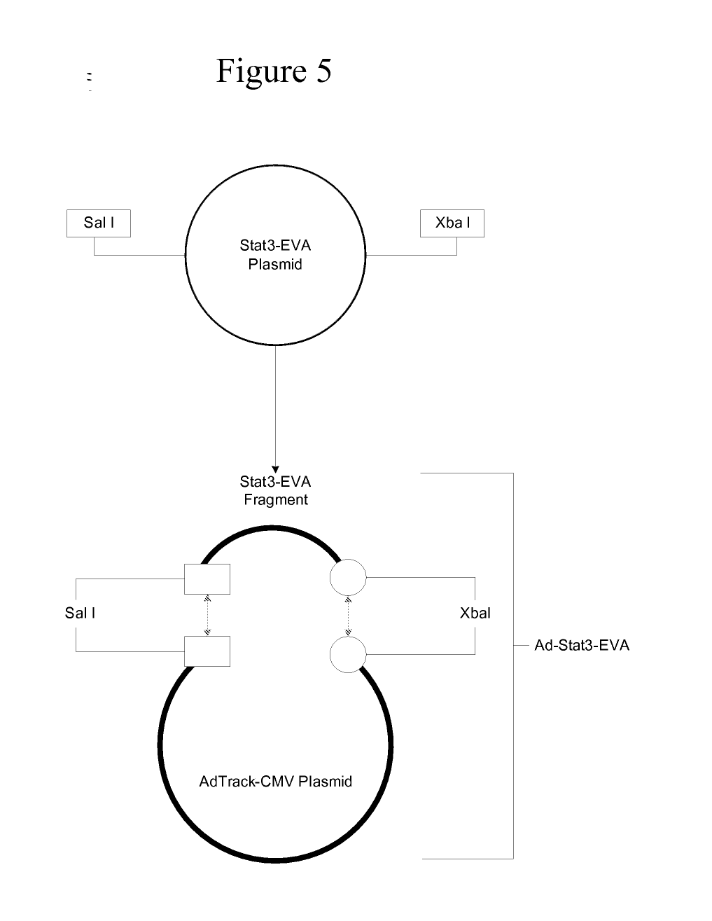

Referring now to FIG. 4, using the mouse Stat3 cDNA sequence, amino acids 434 and 435 were mutated from glutamic acid to alanine, and amino acids 461-463 were mutated from valine to alanine using PCR-based mutagenesis. Ad-Stat3 EVA was constructed by digesting the plasmid contained Stat3-EVA with Sal I and Xba I and ligating this fragment into the AdTrack-CMV plasmid used to construct recombinant adenoviruses as described by (FIG. 5). Viral stocks were created and purified as described previously and virus titers were determined by both an indirect immunofluorescent assay specific for the 72K E2 gene product and a flow cytometric method which titers adenovirus containing green fluorescent protein. Concentrations of adenovirus detailed in each experiment were placed directly into the medium of cells and incubated for the desired times. Viral infection was confirmed by visually observing GFP expression in infected cells.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. An antisense oligonucleotide targeted to a nucleic acid encoding human STAT3 and which modulates expression of human STAT3, said oligonucleotide comprising at least two mutations of the nucleotides in the DNA binding site of STAT3 whereby the at least two mutations prevents binding to DNA without effecting tyrosine phosphorylation or dimerization.

2. The antisense oligonucleotide of claim 1 wherein amino acids 434 and 435 of SEQ. ID NO: 1 are mutated from glutamic acid to alanine.

3. The antisense oligonucleotide of claim 1 wherein amino acids 461 to 463 of SEQ. ID NO: 1 are mutated from valine to alanine.

4. A pharmaceutical compound comprising a therapeutically effective amount of the oligonucleotide of claim 1.

5. The pharmaceutical compound of claim 4 further comprising at least two mutations of the nucleotides in the DNA binding site of STAT3 whereby the at least two mutations prevents binding to DNA without effecting tyrosine phosphorylation or dimerization.

6. The pharmaceutical compound of claim 5 wherein amino acids 434 and 435 of SEQ. ID NO: 1 are mutated from glutamic acid to alanine.

7. The pharmaceutical compound of claim 5 wherein amino acids 461 to 463 of SEQ. ID NO: 1 are mutated from valine to alanine.

8. A method of inhibiting STAT3 function in human non-small-cell lung cancer cell comprising the step of contacting the cell with the pharmaceutical composition of claim 4.

9. The method of claim 8 wherein the pharmaceutical composition comprises at least two mutations of the nucleotides in the DNA binding site of STAT3 whereby the at least two mutations prevents binding to DNA without effecting tyrosine phosphorylation or dimerization.

10. The method of claim 9 wherein amino acids 434 and 435 of SEQ. ID NO: 1 are mutated from glutamic acid to alanine.

11. The method of claim 9 wherein amino acids 461 to 463 of SEQ. ID NO: 1 are mutated from valine to alanine.

12. An antisense oligonucleotide, comprising:
a nucleic acid encoding human STAT3 and which modulates expression of human STAT3; and
an AdTrack-CMV plasmid;
wherein amino acids 434 and 435 of SEQ. ID NO: 1 are mutated from glutamic acid to alanine;
wherein amino acids 461 to 463 of SEQ. ID NO: 1 are mutated from valine to alanine.
